(19) United States
(12) Patent Application Publication
(10) Pub. No.: US 2024/0423830 A1
(43) Pub. Date: Dec. 26, 2024

(54) INTRAUTERINE 3-DIMENSIONAL DEVICE AND SYSTEM THEREOF

(71) Applicant: INTIMO MEDICAL LTD., Nazareth (IL)

(72) Inventors: Lior EREZ, Shoham (IL); Matan SHABAT, EILAT (IL)

(21) Appl. No.: 18/691,980

(22) PCT Filed: Sep. 17, 2022
(86) PCT No.: PCT/IL2022/050997

§ 371 Date: Mar. 14, 2024

(30) Foreign Application Priority Data
Sep. 17, 2021 (IL) 286451

(51) Int. Cl. A61F 6/16; A61B 17/43; A61F 6/18; A61M 25/10

(52) U.S. Cl. CPC A61F 6/16; A61B 17/43; A61F 6/18; A61M 25/10185; A61M 2025/105; A61M 2210/1433

(57) ABSTRACT

An intrauterine device (IUD) useful for various gynecological procedures including fertility treatments, drug delivery to the uterus, contraception. The IUD configured to a compressed small profile tubular configuration and an expanded configuration having a three-dimensional spherical structure. The self-expandable anchor portion of the IUD provided from a slotted tube featuring a plurality of parallel support members.

### FIELD OF THE INVENTION

The present invention relates to intrauterine devices and methods of uses thereof. More specifically, the present invention relates to intra-uterine devices useful for gynecological procedures including infertility treatments, fertility treatments, drug delivery to the uterus and contraception.

### BACKGROUND OF THE INVENTION

IntraUterine Devices (IUDs) are small devices insertable into the uterus for various treatments, for instance, for treating uterine-related disorders, for fertility treatments and for preventing pregnancy.

With regard to pregnancy prevention, the IUDs are the most widely used reversible method of contraception in the world today, particularly in developing countries where it is used by 14.5% of women of reproductive age. In the developed world, this percentage is only 7.6%.

Over the years IUDs intended for various treatments including contraception have been constructed in numerous shapes and sizes and of various materials, some of which are described below:

U.S. Pat. No. 7,080,647 describes a T-shaped intrauterine device which includes a longitudinal branch constituting a body of the T, to the upper higher of which is connected a transversal stem constituting the arms of the T. The body of the T is made up of a fiber that releases an active substance. The fiber constituting the body of the T presents a transverse channel on its upper part. The arms are inserted by force into the channel to form two equal arms on opposite side of the body.

U.S. Pat. No. 3,933,152 describes an intrauterine contraceptive device which is inserted into a uterus in a collapsed position and is held in an expanded position by introduction of a self-hardening liquid plastic into the device after it is positioned within the uterus. In one embodiment the device is provided with an inflatable membrane for positioning it within the uterus prior to insertion of the liquid plastic, after which the membrane is deflated and, in some instances, removed.

U.S. Pat. No. 3,817,248 describes a self-powered device for the continuous and controlled delivery of an agent over a prolonged period of time to an agent receptor is disclosed. The device is comprised of a pressure distendable receptacle formed of an elastic material, with the receptacle communicating with a discharge port having a flow resistive agent metering means for releasing agent in fluid form. The receptacle has an entry port providing access to its interior with the port having a sealing means.

U.S. Pat. No. 4,677,967 describes an intravaginal anchoring device for mammalian females comprised of an elongated tubular body provided with a plurality of slots disposed substantially parallel to each other and to the length of the tubular body such that, in an expanded position, the ends of the body are moved towards each other and flange portions of the body between adjacent pairs of slots are distended away from an axis of the body and, in a collapsed position, the ends of the body are moved away from each other and the flange portions are retracted towards the axis.

WO2018039225 describes a system for treating abnormal muscle activity via energy application includes a catheter, an electrode assembly (106) including a plurality of electrodes (114), a processor configured to receive signals associated with intrinsic tissue activity sensed by the plurality of electrodes, the processor determining a location of a target tissue to be treated based on the sensed intrinsic activity, and a stimulator configured to transmit energy to at least one of the plurality of electrodes located at the target tissue.

US201013144624 discloses an Intra Uterine Ball (IUB) device useful for a gynecological procedure or treatment. The aforesaid device comprises a hollow sleeve for at least partial insertion into the uterine cavity; and an elongate conformable member with at least a portion comprised of shape memory alloy. The elongate member is adapted to be pushed out from a sleeve within the uterine cavity. It is the core of the invention that the elongate member is adapted to conform into a predetermined three-dimensional ball-like configuration within the uterine cavity following its emergence from the sleeve, such that expulsion from the uterine cavity, malposition in the uterine cavity, and perforation of the uterine walls is prevented. U.S. Pat. No. 5,303,717 relates to a device for attaching a contraceptive device to a uterus wall. The attaching device consists of a non-biodegradable wire (10) attached to the contraceptive device, and a retaining means (11) which can be implanted in the tissue of the uterus; this retaining means (11) consists of a non-biodegradable permanent element (12) and a biodegradable temporary element (13), which temporarily provides the retaining means (11) with a tear resistance greater than that of the permanent element (12) alone. The device of the invention is used during the period which immediately follows childbirth.

US20170246027 describes an intrauterine device which includes a wire having a portion capable of forming a three-dimensional (3D) structure. The 3D structure is elastically deformable to a partially collapsed configuration via a crush force larger than a force applied thereto by a relaxed uterine cavity. The three-dimensional structure is also capable of elastically contracting and expanding in response to contraction and expansion of the uterine cavity.

As described above, multiple designs of IUDs are available nowadays for multiple gynecological procedures. However, if we consider contraception, the most common IUD today is the T-shaped IUD, a copper T-shaped IUD which constantly releases a small amount of copper into the uterus or a hormonal T-shaped IUD which slowly releases a hormone (progestogen) into the uterus.

The first T-shaped IUD was designed more than 30 years ago, and although the device has been improved over the years, the T-shaped IUDs available today still involve several problems and risks as described below:
Expulsion out of the uterus. Expulsion of the T-shaped IUD leads to decreased contraceptive efficacy. It is a relatively common complication occurring in up to 10% of patients-about 2 to 10 out of 100 T-shaped IUDs are expelled from the uterus into the vagina during the first year. Expulsion is more likely to occur when the T-shaped IUD is inserted right after childbirth or in women who have not carried a pregnancy or are 20 years old or younger; (Expulsion rate range for several models of T-shaped IUD based on literature review is: 5.7%-13%).
Displacement of the T-shaped IUD from its proper position next to the uterine fundus. Displacement of the IUD leads to decreased contraceptive efficacy. It occurs in up to 25% of females with an IUD, and in some cases, it involves with cramping or bleeding.
An IUD which is not positioned correctly or migrates out of its optimal position during use can be less effective in preventing a pregnancy. If pregnancy does occur, the presence of the IUD increases the risk of miscarriage, particularly during the second trimester. Removal of the IUD at the beginning of the pregnancy still carries a risk for premature delivery.
Embedment and Perforation of the uterus wall is variable in extent and symptomatology, ranging from embedment in the myometrium to complete transuterine perforation with migration of the IUD into the peritoneal cavity. Embedment of the IUD into the endometrium or myometrium can usually be managed in the outpatient clinical setting but occasionally requires hysteroscopic removal. It may occur to some degree in up to 18% of females with an IUD. Uterine perforation is a serious complication in females with an IUD, occurring in up to one of every 1,000 cases.
Complete uterine perforation, in which the IUD is partially or completely within the peritoneal cavity, requires surgical management.
Pain feeling for the patient during insertion and/or removal.
Bleeding & Cramping is a key side effect leading to IUD discontinuation; (removal rate range for several models of T-shaped IUD based on literature review is: 10.3%-29.6%).
T-shaped IUD loading activity is required prior to insertion, once the sterile package is already open, so there is a risk of infection from bacteria getting into the uterus (PID=Pelvic Inflammatory Disease).
T-shaped IUD deployment requires two-handed operation of the insertion tube and insertion rod.
lost T-shaped IUD strings into the uterine cavity, so an intervention is required for removal using tools, ultrasound, or X-ray (inconvenience to patient).

Other common IUDs include the devices described in U.S. Pat. No. 5,303,717 and US20170246027. The device described in U.S. Pat. No. 5,303,717 consisting of a non-biodegradable wire which is implanted in the tissue of the uterus involves the above describes problems and risks, mainly the risk of expulsion and potentially perforation with the sharp tip of its stylet; (Expulsion rate: 7.6% and removal rate: 15%).

The device described in US20170246027 which includes a wire having a portion capable of forming a three-dimensional (3D) structure. However, such a 3D structure comprises two free ends and therefore forms a non-closed structure that lends itself to expulsion as it is susceptible to the forces applied by the uterus on the device itself, rendering it susceptible both to expulsion and tissue perforation [Wiebe and Trussell, Contraception. 2016 April; 93 (4): 364-366.] Such device involves the above describes problems and risks and mainly the risk of expulsion as the risk of expulsion is greater with resilient devices than with more rigid devices; (Expulsion rate: 5.3% and removal rate: 23.8%).

### SUMMARY OF THE INVENTION

The present invention overcomes these deficiencies of the background by providing an intrauterine device that has a closed form three-dimensional configuration that is more adept at handling forces applied by the uterine environment.

Embodiments of the present invention provide, an IUD device which can be utilized for many applications for example including contraception, treat uterine-related disorders, insemination, fertility treatments, uterine local drug delivery, uterine monitoring, and the like.

In embodiments, the IUD device according to the present invention may be used in a safe and comfortable manner over a selective period of time, for example, the device may be used for a short period for undertaking a treatment, alternatively, the device may be used for an extended period of time as a contraceptive device.

In embodiments of the present invention provide an intrauterine 3-dimensional device for reversible contraception and/or for active agent delivery and/or fertilization of a human or animal female, the device comprises:
a self-expandable anchor having a 3-dimensional shape to obtain peripheral retention against the uterus wall;
wherein when pushed by uterus forces, the self-expandable anchor resist to expulsion, and
wherein when pulled, the self-expandable anchor is collapsed to become narrow and allow removal via cervical canal;
a rounded distal tip connected to a distal end of the self-expandable anchor to minimize pain during insertion into the uterus and/or to reduce risk for the uterine wall to be perforated;
a string connected to a proximal end of the self-expandable anchor to enable pulling and removal out of the uterus.

In embodiments, the intrauterine 3-dimensional device may be configured to further comprise:
an elastic balloon enclosed by the self-expandable anchor; the balloon may provide peripheral support to the self-expandable anchor to further prevent the anchor from collapsing;
a catheter, the balloon is connected to a distal end of the catheter;
a sealing septum connected to a proximal end of the catheter; and
a dripping cannula connected to a distal outlet of the elastic balloon, so that when the balloon is inflated with fluid, it functions as a pump to drip liquid droplets inside the uterine cavity.

In embodiments of the present invention, the self-expandable anchor is comprised of multiple ribs.

In embodiments of the present invention, the self-expandable anchor may be configured in the form of a slotted-tube made of super-elastic and/or shape-memory material

In embodiments, the super-elastic and shape-memory material may for example include but is not limited to a nickel-titanium alloy, shape-memory polymers, any combination thereof or the like.

In embodiments of the present invention, the intrauterine 3-dimensional device may be configured to further comprise at least one or more active agent(s) for example including but not limited to at least one or more of copper, hormone reservoir, chemical substance, biological substance, drug, medicine, any combination thereof or the like.

In embodiments, the active agent of copper may either at least one copper sleeve and/or copper bead. Optionally, active agent of copper may be configured to have a surface area of 150 mm^2 (millimeters squared) to 380 mm^2 (millimeters squared).

In embodiments, the intrauterine 3-dimensional device may further comprise at least one of a distal stopper and/or a proximal stopper. In embodiments, the distal stopper may be made of metal, alloy, or a polymeric material. In embodiments, the proximal stopper may be made of Silicone, metal or a polymeric material.

In embodiments, the compliant balloon may be adhered to the catheter and covers an eyelet featured at the distal end of the catheter.

In embodiments, the balloon may be inflated and/or filled with a sterile fluid through a proximal end of the catheter and at the distal end through an eyelet.

In embodiments, the balloon may be made of flexible polymeric material for example including but not limited to at least one or more materials selected from: Polyurethane, Nylon, PET, EVA membrane or elastomeric material selected from Silicone, Silicone membrane (polydimethylsiloxane), Polyurethane, any combination thereof or the like.

In embodiments, the catheter may comprise a flexible shaft made of flexible polymeric material.

In embodiments, the proximal stopper is adherable to the catheter.

In embodiments, the dripping cannula may be an extruded flexible tube made of thermoplastic elastomer for example including but not limited to at least one or more materials selected from the group comprising of Pebax, Polyurethane, Polyethylene, any combination thereof, or the like.

In embodiments, the lower half of the self-expandable anchor may be coated by a thin layer seal. Optionally, the thin layer seal is designed as an inverted umbrella made of elastomeric material selected from Silicon, and polymeric material(s).

In embodiments, the sealing septum may be configured to prevent the catheter from being drawn into and/or pulled into a uterine cavity.

In embodiments, the intrauterine 3-dimensional device may further comprise at least one or more sensor and/or biosensor for monitoring biophysical parameters in utero. Such a sensor functionally associated with and/or integrated with the IUD device of the present invention may for example be provided in the form for example including but not limited to at least one or more of: a pH sensor, a temperature sensor, a pressure sensor, an electrolytes sensor, and oxygen concentration sensor, the like or any combination thereof.

In embodiments, there is provided an intrauterine device insertion kit comprising
the intrauterine 3-dimensional device according to embodiments of the present invention and an insertion tool. Optionally the kit may further comprise a septum sealing member and a corresponding septum applicator tool for coupling the septum sealing member.

In embodiments, the IUD inserter tool may be comprised of a handle, an insertion tube, a plunger, and a flange. Optionally, the insertion tube, the plunger and the flange may be provided from optional materials selected from metal, alloy, or polymeric material(s), or any combination thereof.

In embodiments, the distal part of the insertion tube is marked on its external surface with a graduation scale and/or ruler scale to indicate an insertion depth into the uterus.

In embodiments, the septum applicator tool may comprise a handle, tracks, flexible arms, self-sealing septum, cavity for septum, integral hinge, funnel shape cannula, and slider. In embodiments, the handle of the septum applicator may be made of material selected from metal, alloy, or polymeric material(s).

In embodiments, the self-sealing septum may be made of an elastomer selected from Silicone and polymer(s).

In embodiments, the slider of the septum applicator tool may be made of metal, alloy, or a polymeric material.

In embodiments, the funnel shape cannula of the septum applicator tool may be functionally coupled and/or connected to the slider via welding, gluing, mechanical snap-fit or any other way.

In accordance with some embodiments of the present invention, the key components of the intrauterine 3-dimensional device which allow its robust functionality are described as follow:
a self-expandable multiple ribs anchor made of a super elastic material. When the self-expandable multiple ribs anchor is radially deployed inside the uterus, it has a 3-dimensions (3D) spherical shape to obtain peripheral retention against the uterus wall to prevent displacement and migration of the device. When pushed by uterus axial force, the anchor ribs are axially collapsed to become wider and resist expulsion, but when controllably pulled under axial force, the ribs are radially collapsed to become narrow and allow removal via cervical canal.
an internal small balloon enclosed by the self-expandable multiple ribs anchor and connected to a dripping flexible cannula. The balloon while inflated inside the uterus, obtains peripheral mechanical support to the multiple ribs to prevent anchor collapse due to axial and radial forces applied by the uterus and so resist to expulsion of the device out of the uterus. Following use the balloon may be deflated, the ribs may be controllably collapsed by applying a pulling force on the catheter, so intrauterine 3-dimensional device can be removed out of the uterus.

In accordance with some embodiments of the present invention, the self-expandable anchor made of super-elastic material is deployed first and obtain mechanical retention inside the uterus. Under axial pushing force the self-expandable anchor becomes wider so has a good resistance for expulsion, but under radial force it becomes narrow so has partial resistance for expulsion; thus, when balloon catheter is implemented for fluids delivery the internal balloon may be filled with a sterile fluid to add mechanical support to the ribs and obtain non-collapsible anchor during treatment administration.

When the intended use of the intrauterine 3-dimensional device is long-acting reversable contraception, a rounded distal tip of a ball such as a copper or hormone ball is connected to the distal end of the self-expandable multiple ribs anchor to minimize pain feeling during the insertion procedure. The distal copper/hormone ball is designed to be out of the insertion tube to minimize irritation with the cervix canal due to smooth surface and rounded shape.

In addition, the rounded shape anchor obtains 360-degree support to eliminate embedment and perforation of the uterus wall.

When intended for short-term fluids delivery into the uterus, the intrauterine 3-dimensional device is slightly modified, i.e., the elastic balloon's distal end is connected to a flexible cannula and the balloon's wall may be drilled for some micro-holes and/or micro-pores by laser or similar means, so when balloon is inflated with a fluid (for example including but not limited to sperm, washed sperm, drug, active agent, biologics, hormones, medicament, any combination thereof or the like), it functions as a small volume elastomeric pump to drip liquid's droplets locally inside the uterus in a slow-release manner.

The flow rate pre-defined and controlled by optional cannula parameters for example including but not limited to at least one or more selected from inner diameter, length, friction, friction coefficient, number of micro-holes, number of micro-pores, size of micro-holes, size of micro-pores, pore diameter, pore shape, any combination thereof or the like. The cannula in communication with balloon's pressurized volume and functions as a flow restrictor in elastomeric pump. The lower half of the self-expanding anchor may be covered with a thin layer of elastomeric sealant, so when deployed next to internal OS it obtains sealing against the uterus wall and the released droplets cannot leak out of the uterus via cervical canal.

The intrauterine 3-dimensional device may be used in a safe and comfortable manner as it is supplied sterile, pre-loaded and ready to use without manual handling of the device after the sterile pack was opened, thus, the risk for contamination and infection is minimal. The intrauterine 3-dimensional device is inserted into the uterus by one-handed intuitive inserter for deployment in about 5-10 sec.

Unless otherwise defined, all technical and scientific terms used herein have the same meaning as commonly understood by one of ordinary skill in the art to which this invention belongs. The materials, methods, and examples provided herein are illustrative only and not intended to be limiting.

Implementation of the method and system of the present invention involves performing or completing certain selected tasks or steps manually, automatically, or a combination thereof.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic block diagram of an exemplary device, kit and system according to embodiments of the present invention;

FIG. 2A-C are schematic illustration showing various views of a portion of an exemplary device according to the present invention; FIG. 2A shows a perspective view of an exemplary anchor portion of the device in its closed and/or small profile configuration; FIG. 2B shows a perspective view of an exemplary anchor portion of the device in its open and/or expanded profile configuration; FIG. 2C shows a top-down view of the device portion shown in FIG. 2B;

FIG. 3 shows a perspective view of a schematic illustration of an exemplary IUD device according to embodiments of the present invention;

FIG. 4A-B show schematic illustration of an exemplary IUD device according to embodiments of the present invention; FIG. 4A shows a perspective view of an exemplary IUD device; FIG. 4B shows an exploded view of FIG. 4A;

FIG. 5A-B show schematic illustration of an exemplary IUD device according to embodiments of the present invention; FIG. 5A shows a perspective view of an exemplary IUD device; FIG. 5B shows an exploded view of FIG. 5A;

FIG. 6A-C are schematic illustration showing various views of a portion of an exemplary IUD device according to embodiments of the present invention; FIG. 6A shows a perspective view of an exemplary anchor portion of the device in its open and/or expanded profile configuration; FIG. 6B shows a top-down view of the device portion shown in FIG. 6A; FIG. 6C shows a perspective view of an exemplary anchor portion of the device in its closed and/or small profile configuration;

FIG. 7A shows a schematic illustration of two exemplary IUD device following deployment within the uterus, according to embodiments of the present invention;

FIG. 7B shows a schematic illustration of two exemplary IUD device following deployment within the uterus, according to embodiments of the present invention;

FIG. 8A-B show schematic illustration of an exemplary IUD insertion device according to embodiments of the present invention; FIG. 8A shows a perspective view of an exemplary IUD insertion tool; FIG. 8B shows an exploded view of FIG. 8A;

FIG. 9A-C show schematic illustrations of an exemplary IUD device in use and association with an IUD insertion device according to embodiments of the present invention; FIG. 9A shows a partial view of an exemplary IUD insertion tool associated with an IUD device according to embodiments of the present invention;

FIG. 9B shows a partial view of an exemplary IUD insertion tool associated with an IUD device according to embodiments of the present invention; FIG. 9C shows a partial view of an exemplary IUD insertion tool associated with an IUD device according to embodiments of the present invention;

FIG. 10A-B show schematic illustrations of an exemplary IUD device during its deployment within the uterus with an IUD insertion device, according to embodiments of the present invention; FIG. 10A shows the insertion of the IUD device; FIG. 10B shows the deployment and/or delivery of the IUD device;

FIG. 11 shows a schematic illustrations of an exemplary IUD device following deployment within the uterus, according to embodiments of the present invention;

FIG. 12 shows a schematic illustrations of an exemplary IUD device during use within the uterus, according to embodiments of the present invention;

FIG. 13A-C show various view of a schematic illustration of an exemplary sealing septum applicator tool according to embodiments of the present invention; FIG. 13A shows a perspective view of an exemplary sealing septum applicator tool; FIG. 13B show a close up view of an end of an exemplary sealing septum applicator tool; FIG. 13C shows a partial exploded view the sealing septum applicator tool shown in FIG. 13A; and

FIG. 14A-E show various view of a schematic illustration of an exemplary sealing septum applicator tool in use with an IUD device according to embodiments of the present invention.

### DESCRIPTION OF THE PREFERRED EMBODIMENTS

Embodiments of the present invention provides a unique intrauterine device having 3-dimensional shape. The device is configured to be used both as a contraceptive device and/or as a device for active agent delivery and/or insemination of a human or animal female

As described above embodiments of the present invention overcomes the problems, for example spontaneous migration and/or expulsion, and further prevents risks, for example perforation, associated with current available IUDs such as T-shaped IUDs, frameless IUDs, and ball-shaped IUDs.

In accordance with some embodiments of the present invention, the intrauterine 3-dimensional device provides a robust device capable of being utilized as a long-acting reversible contraception device and/or an active agent delivery device and/or an insemination device for inseminating a human or animal female.

In embodiments, the IUD device according to embodiments of the present invention may be utilized as a localized uterine active agent delivery system comprising a self-expandable anchor comprising multiple ribs provided from super elastic materials and a balloon member disposed therein that is further functionally coupled to catheter and dripping cannula, for the delivery of the active agents. In embodiments the IUD device may further be functionally coupled to a sealing member to facilitate the localized active agent delivery.

In some embodiments of the present invention, wherein the IUD device is intended for use as a contraception device, the IUD device may comprise a self-expandable anchor, configured to be deployed within the uterine cavity, and further featuring a retrieval member(s). In some embodiments the IUD device of the present invention may further comprise and/or associated with active contraceptive agents for example including but not limited to copper, and/or hormones and/or medicaments or the like agents that contraceptive agents, for local delivery. As is known in the art such contraceptive agents may be provided in the form of a copper sleeves, a copper coated substrate, hormone reservoir, copper beads, hormone beads, medicaments.

The principles and operation of the present invention may be better understood with reference to the drawings and the accompanying description.

The following figure reference labels are used throughout the description to refer to similarly functioning components are used throughout the specification hereinbelow.

10 auxiliary devices;

50 system;

55 IUD kit;

100,200,800 self-expandable intrauterine device;

102,212 distal body portion;

104,206,810 tubular self-expanding body portion;

104a,902 rib/support members/anchoring members;

104c,812 coating;

104v internal volume;

106,204,814 proximal body portion;

108202, retrieval member;

110,208,210 treatment module;

120 sensor module;

130 agent delivery channel assembly;

132,802 distal channel;

134,806 expandible bladder and/or balloon;

136,804,816 proximal channel;

138,822 sealing member;

140,1600 sealing member introducing tool;

150,1100 device introducing tool;

800 intrauterine 3-dimensional device;

802 flexible dripping cannula;

804 balloon catheter;

806 balloon;

808 eyelet;

810 self-expandable anchor;

812 thin layer seal;

814 proximal stopper;

816 self-expandable anchor tube;

818 catheter distal outlet;

820 distal opening;

822 self-sealing septum;

824 sealing member lumen/septum canal;

826 sealing member proximal end/filling port;

1100 inserter;

1102 handle;

1102A lower slider;

1102B upper slider;

1104 insertion tube;

1106 flange

1202 plunger;

1600 septum applicator/seal introducing tool;

1602 handle;

1604 male/female tracks

1604A female tracks;

1604B male tracks;

1606 flexible arms;

1606A first flexible arm;

1606B second flexible arm;

1606C flexible arms protruding teeth/holding member;

1610 funnel shaped cannula internal hinge;

1612 funnel shape cannula;

1612A, first half/portion of funnel shaped cannula;

1612B second half/portion of funnel shaped cannula;

1612C proximal channel/tubular shaped portion;

1612D distal funnel shaped portion;

1614 slider housing member;

1616 cavity/seal receiving recess;

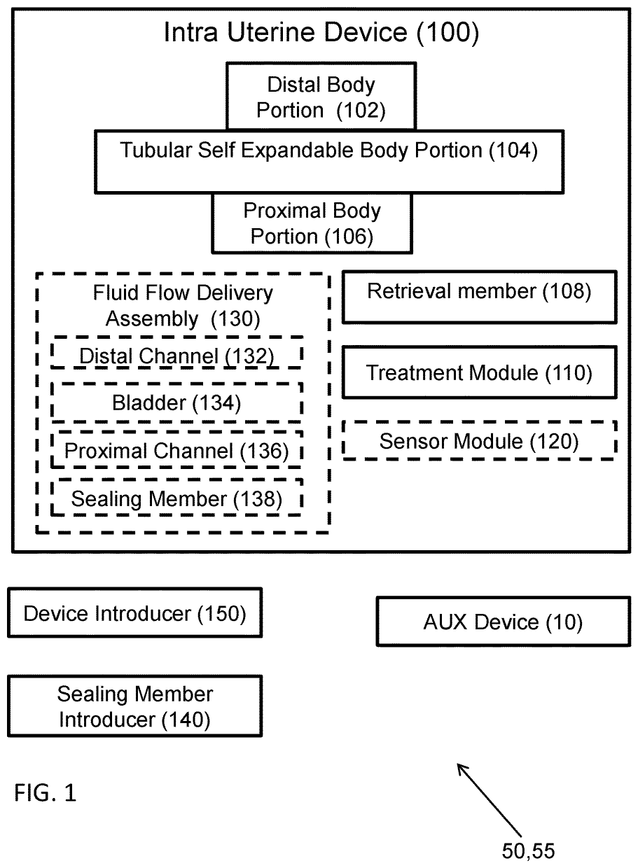

Referring now to the drawings, FIG. 1 shows a schematic box diagram of an Intrauterine Device (IUD) 100, a system 50 and kit 55 thereof according to embodiments of the present invention, wherein device 100 is configured for placement within the uterus. Device 100 has a body that is configured to be self-expandable so as to ensure that the device remains within the uterus and does not independently and/or spontaneously expel and/or exits the uterus. As such device 100 is configured such that the forces acting on the device 100 from the uterus do not negatively affect its placement and/or functionality and/or stability within the uterus.

Device 100 comprises a three-dimensional anchoring structure having a continuous external enveloping surface that is devoid of free ends. In embodiments, the anchoring structure comprises a distal body portion 102, a tubular self-expanding body portion 104 and a proximal body portion 106. Most preferably device 100 further comprises a retrieval member 108 and at least one or more treatment module 110.

In some embodiments, device 100 may further comprise an agent delivery assembly 130, as is shown in broken line,

In some embodiments, device 100 may further comprise a sensor module 120, as is shown in broken line,

In some embodiments, device 100 or any portion thereof in particular at least a portion of distal body portion 102 and/or anchoring body member 104 and/or proximal body portion 106 may be provided with a coating 104c provided from optional materials. For example, a such a coating may be provided in the form of a medicament for facilitating drug delivery. For example, coating may be provided in the form of a sealant, for example to prevent fluid flow leakage. For example, such a coating may be provided in the form of a high surface area and/or friction surface so as to increase interface and/or friction with the surrounding tissue.

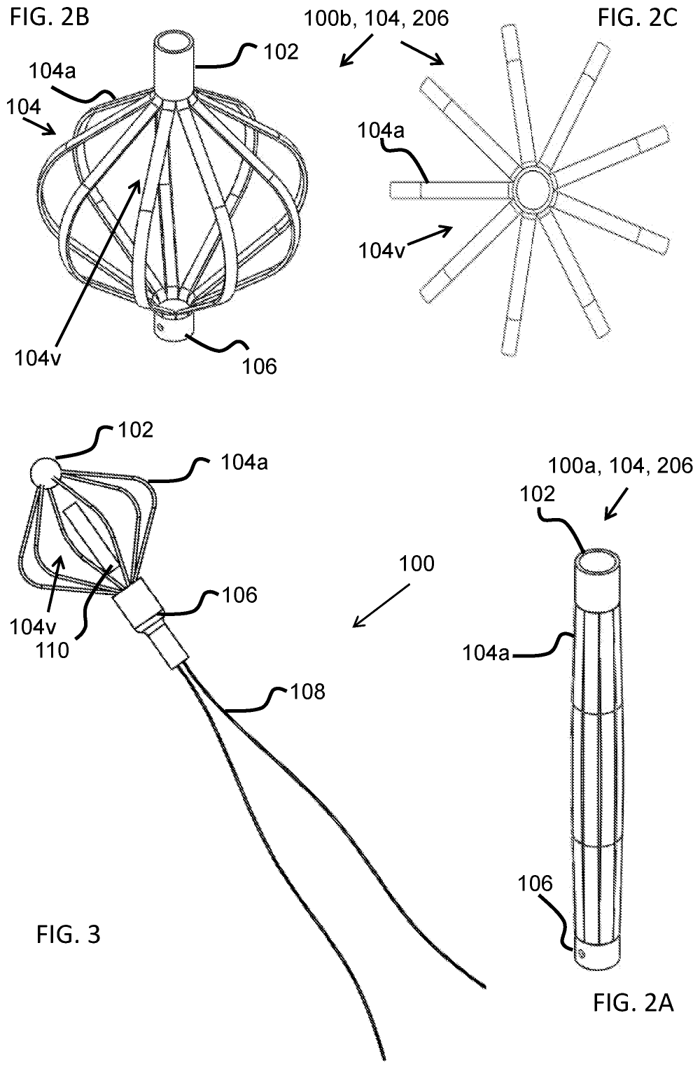

In embodiments the tubular self-expanding body portion 104 that is configured to assume at least two interchangeable configurations including a small profile tubular configuration 100a, a non-limiting example of which is shown in FIG. 2A, and an expanded configuration 100b, a non-limiting example of which is shown in FIG. 2B.

In embodiments, self-expanding body portion 104 is provided from super-elastic and shape-memory materials that are configured to have at least two interchangeable configurations, an expanded configuration 100b and a small profile configuration 100a, for example as shown in FIG. 2A-C.

In embodiments portions of device 100 and in particular self-expanding body portion 104 may be provided from materials for example including but not limited to comprising the group selected from at least one of: polymers, alloys, smart materials, shape memory materials, nitinol (a nickel-titanium alloy), materials exhibiting plastic deformation, super-elastic metal alloy which transforms from an austenitic state to a martensitic state, or any combination thereof.

In embodiments, self-expanding body portion 104 may optionally be provided in the form of a plurality of flanking anchoring members 104a disposed between distal body member 102 and proximal body member 106. Preferably in the small profile configuration 100a flanking anchoring members 104a are arranged adjacent to one another in a radial manner, along their long axis and/or dimension, forming a tubular and/or pipe like configuration.

Optionally anchoring members 104a may be arranged in parallel to one another along the long axis.

Optionally anchoring members 104a may be arranged adjacent to one another between distal body member 102 and proximal body member 106 such that the long axis of anchoring members 104a at an angle of about 90 degrees relative to both distal body member 102 and proximal body member 106.

Optionally anchoring members 104a may be arranged adjacent to one another between distal body member 102 and proximal body member 106 such that the long axis of anchoring members 104a form an acute angle relative to at least one of distal body member 102 or proximal body member 106.

Preferably, anchoring members 104a are configured to be flexible and/or compliant along their long axis.

In embodiments, self-expandable body 104 may be configured in the form of a slotted tube, therein the anchoring members 104a, distal body member 102 and proximal body member 106 may be configured from a single unitary material.

In embodiments the small profile configuration 100a is utilized during deployment or insertion and during removal of device 100 into or out of the uterine cavity. Small profile tubular configuration comprises a minimal volume of device 100 wherein the radial diameter of the device is minimized.

In embodiments small profile configuration 100a may be configured to have a delivery diameter of up to about 3 mm and more preferably up to 2.5 mm.

In embodiments the expanded profile configuration 100b is utilized during use within the uterus and may be used as a short-term device, for example for as a treatment device, an insemination device, drug delivery device or the like; or device 100 may be utilized as long-term device, for example as a reversible contraceptive device. Expanded profile configuration 100b comprises a maximal volume of device 100 having an outer diameter of at least 12-15 mm. Preferably, the expanded profile 100b is configured such that it does not spontaneously exit the uterine body. In embodiments, the internal volume 104v of device 100 in its expanded profile configuration 100b is up to about 3 cm^3 (centimeters cubed) and more preferably up to about 1.5 cm^3 (centimeters cubed).

In embodiments, proximal body portion 106 and distal body portion 102 provide a distal and proximal end member of the anchor portion of device 100. Optionally and preferably each of the distal body portion 102 and proximal body portion 106 provide an end ensuring a continuous enveloping surface for anchoring portion of device 100.

In embodiments, distal body portion 102 may be configured to receive and/or associate and/or couple with additional members and/or devices. For example, distal body portion may be configured to receive at least one or more of a treatment module 110 and/or a drug delivery channel and/or drug delivery catheter and/or an agent delivery assembly 130, a combination thereof or the like.

In embodiments, proximal body portion 106 is preferably configured to functionally associate with and/or receive a retrieval member 108. may be configured to receive and/or associate and/or couple with additional members and/or devices. For example, proximal body portion may be configured to receive at least one or more of a treatment module 110 and/or a drug delivery channel and/or drug delivery catheter, and/or an agent delivery assembly 130, a combination thereof or the like.

In embodiments, retrieval member 108 may be configured to facilitate removal of device 100 from within the uterus. In embodiments retrieval member 108 may be provided to facilitate collapsing and/or transitioning device 100 from its expanded, large diameter, configuration 100b to its small profile, small diameter, configuration 100a, therein facilitating removal of device 100 from its position within the uterus. In embodiment retrieval member 108 may be configured to apply the necessary forces and/or manipulation to allow for the transition of device 100 from expanded configuration 100b to small profile configuration 100a.

In embodiments, treatment module 110, may be associate with any portion of device 100 for example including but not limited to distal body portion 102, proximal body portion 106, self-expandable body portion 104, an internal portion and/or surface 104i of expandable body portion 104, an external portion and/or surface 104e of expandable body portion 104, retrieval member 108, a combination thereof or the like.

Treatment module 110 preferably provides for utilizing IUD device 100 for introducing a medicament, agent, hormone, copper, fertility agent, contraceptive agent, for local delivery to any portion of the female reproductive tract and in particular the uterus and the uterine cavity. Treatment module 110 may be provided in optional forms for example including but not limited to copper beads, copper loops, hormones, medicaments, agents, contraceptive agents, fertility agents, copper sleeves, a copper coated substrate, hormone reservoir, hormone beads, any combination thereof or the like.

In embodiments sensor module 120 may for example be provided in the form for example including but not limited to at least one or more of: a biosensor, a pH sensor, a temperature sensor, a pressure sensor, an electrolytes sensor, and oxygen concentration sensor, the like or any combination thereof. In embodiments sensor module 120 is preferably utilized to introduce at least one or more sensor and/or biosensor for monitoring biophysical parameters in utero.

In embodiments sensor module 120 may be associate with any portion of device 100 with any portion of device 100 for example including but not limited to distal body portion 102, proximal body portion 106, self-expandable body portion 104, an internal portion and/or surface 104i of expandable body portion 104, an external portion and/or surface 104e of expandable body portion 104, retrieval member 108, a combination thereof or the like.

In embodiments, sensor module 120 of device 100 may be in communication with at least one or more auxiliary device 10, therein forming a system 50. In embodiments auxiliary device 10 may for example include but is not limited to at least one or more of: smartphone, mobile processing and communication device, imaging device, server, display, computer, healthcare service provider dedicated system, first respondent call center, health care call center, the like or any combination thereof. In embodiments sensor module 120 may be in communication and/or have a wired and/or wireless data exchange link with at least one or more auxiliary device 10. In embodiments communication and/or data exchange link between sensor module 120 and optional auxiliary devices may be provided utilizing wired and/or wireless and/or contactless, communication protocols as is known in the art for example including but not limited to wireless communication protocols, cellular communication, wired communication, near field communication, BLUETOOTH, ZIGBEE, optical communication, Radio Frequency communication (RF), the like and/or any combination thereof.

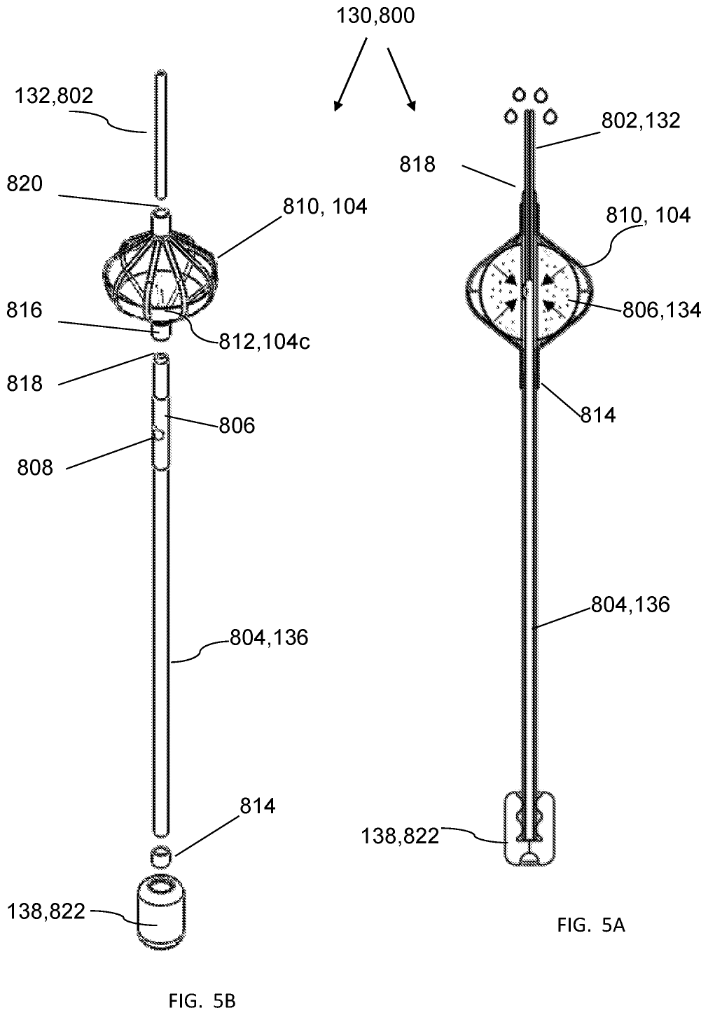

In embodiments, IUD device 100 may be functionally associated with an agent delivery assembly 130, a non-limiting example of which is depicted in FIG. 5A-B. Agent delivery assembly 130 may be associated with device 100 about at least a portion of distal body portion 102, proximal body portion 106, and self-expandable body portion 104 and more preferably an internal volume of the self-expandable body portion 104.

In embodiments, agent delivery assembly 130 comprises a distal delivery channel and/or catheter 132, a compliant balloon and/or bladder 134, a proximal channel and/or catheter 136 and a sealing member 138.

In embodiments, agent delivery assembly 130 provides for delivery of a flowing fluid through the distal delivery channel 132, for example including but not limited to medicament, hormone, saline, washed sperm, fertilization fluid, sperm, drug, biologics, insemination fluid, contraceptive fluid, any combination thereof or the like flowing fluid that may be delivered into the uterine cavity.

Preferably compliant balloon and/or bladder 134 is most preferably associated within an internal volume formed by self-expandable body portion 104 such that the maximal volume of balloon 134 is configured to fit within the internal volume provided by body portion 104.

Preferably compliant balloon and/or bladder 134 features a through channel formed from distal channel 132 and proximal channel 136, allowing for delivery of a flowing fluid from a source external to the uterus to be introduced into the uterine cavity, preferably introduced via the proximal channel 136 and delivered and/or dispensed through distal channel 132.

Preferably distal delivery channel 132 is associated with distal body portion 102.

Preferably proximal channel 136 is associated with both distal body portion 102 along the internal volume of body portion 104. In embodiment proximal channel 136 may be provided in the form of balloon catheter for controlling the volume of bladder and/or balloon 134.

In embodiments a sealing member 138 may be utilized to seal a proximal portion of proximal channel 136. Preferably sealing member 138 is associated with proximal channel 136 about a proximal end thereof with a dedicated tool 140.

In embodiments sealing member 138 may comprise a port and/or recess 138a for introducing a flowing fluid into proximal channel 136.

In embodiments, the present invention provides a system comprising an IUD device 100 that is in communication with optional auxiliary devices 10 as previously described.

In some embodiments, the present invention provides a kit 55 comprising a at least one IUD device 100 and at least one dedicated IUD tool introducer 150 utilized to delivery and/or deploy and/or introduce the IUD 100 within the uterus.

In embodiments a vaginal speculum and/or medical imagery devices, for example including but not limited to ultrasound, as are known in the art, may be utilized along with a dedicated introducer tool 150 to facilitate delivery of the device 100 into the uterus and the uterine cavity.

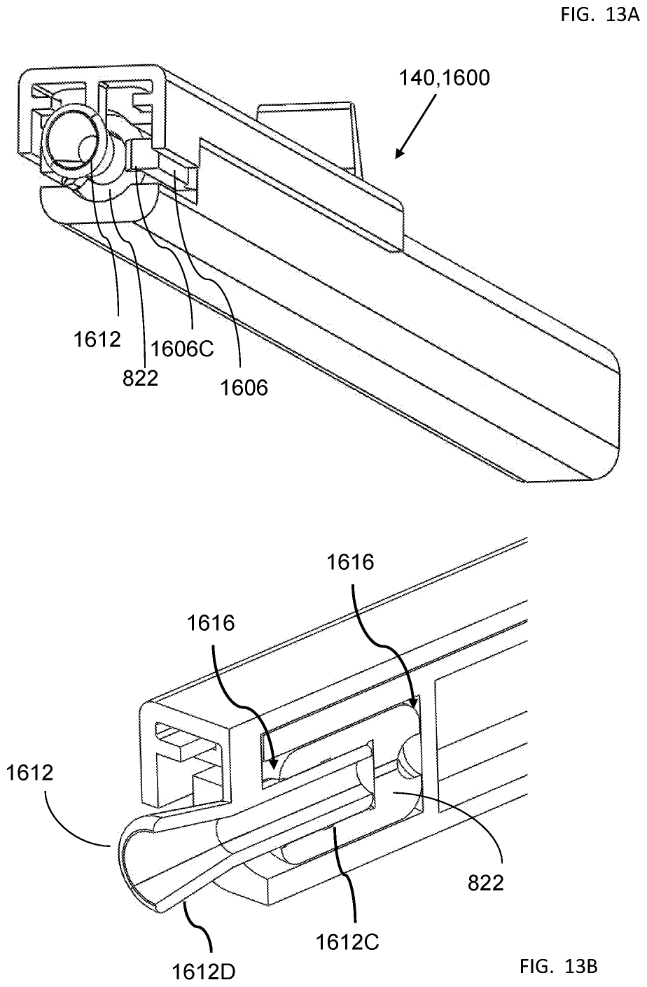

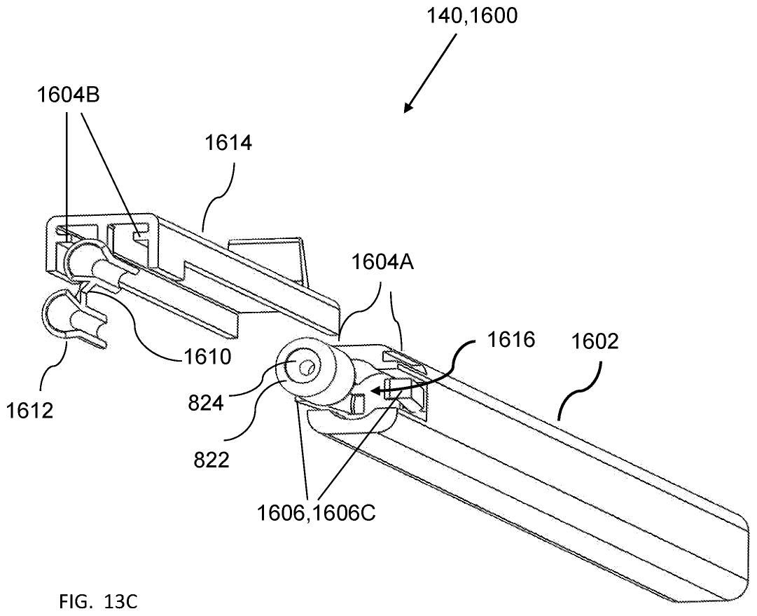

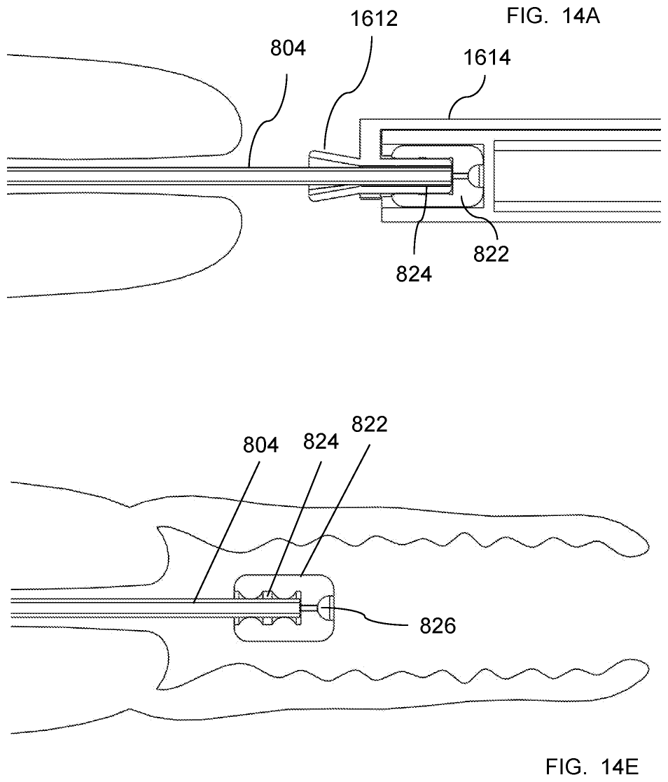

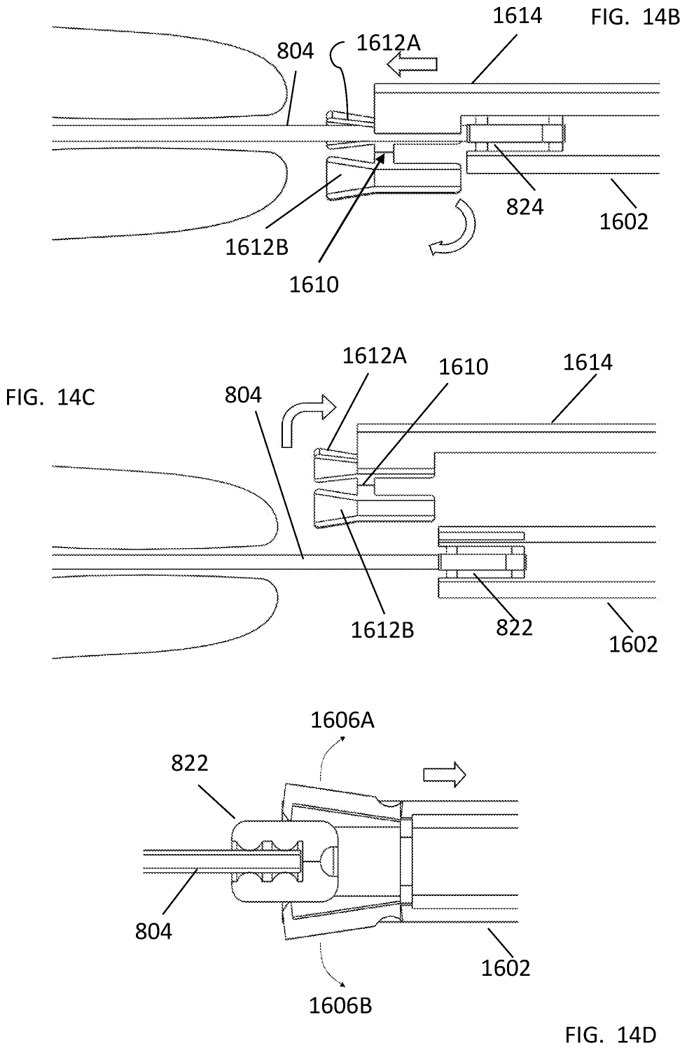

In embodiments, kit 55 may further comprise at least one or more sealing members 138 and a dedicated sealing member tool 140, described in greater detail in FIGS. 13-14, configured for introduce the sealing member 138 onto a portion of device 100, and in particular a proximal channel 136 of agent delivery assembly 130.

FIG. 2A-C show various views of a non-limiting exemplary configuration of the tubular self-expandible body portion 104,206 of IUD device 100, according to embodiment of the present invention.

FIG. 2B is a perspective view of the self-expandable anchor 104,206 shown in the expanded configuration 100b. FIG. 2C shows top view of the self-expandable anchor 104,206 in an expanded configuration 100b.

FIG. 2B-C show anchor member 104,206 having a lantern-like configuration comprising a plurality of flexible flanking anchoring members 104a disposed between distal body member 102 and proximal body member 106. The expanded configuration 104b having an ovoid lantern like configuration wherein individual anchoring members and/or ribs 104a extend between distal body portion 102 and proximal body portion 106.

FIG. 2A illustrates the self-expandable anchor 104,206 in its a collapsed and/or small profile configuration 100a.

In embodiments of the present invention, the self-expandable anchor 206 may be a slotted-tube made of super-elastic and shape-memory material such as, for instance, Nitinol (NiTi; a nickel-titanium alloy). The slotted-tube may undergo heat treatment to form the final shape of the self-expandable anchor 206, e.g., a 3-dimensional multiple ribs 104a sphere, a non-limiting example of which is seen in FIGS. 2B-2C.

FIG. 3 shows a perspective view a non-limiting exemplary configuration of IUD device 100 according to embodiment of the present invention. Device 100 featuring a distal body portion 102, a proximal body portion 106 and a self-expanding body portion 104.

Device 100 further features a treatment module 110, disposed within the internal open volume 104v of body portion 104. In embodiments a treatment module 110 may be disposed within the volume 104v so as to prevent and/or limit the treatment module's interaction with tissue. For example, a treatment module 110 provided in the form of a copper sleeve disposed internal to volume 104v may be configured so as to prevent and/or limit direct contact and/or interfacing between the copper sleeve and uterine tissue most preferably the endometrium.

Device 100 further features a retrieval member 108 extending proximally to proximal body portion 106. Retrieval member 108 is configured to facilitate intended and/or controlled removal of device 100 from the uterus. Most preferably, retrieval member 108 provides for collapsing body portion 104 from the expanded configuration 100b, lantern-like configuration as shown to the small profile tubular configuration 110a, not shown here. Preferably, body portion 104 is configured to collapse to assume the minimal profile configuration 100a when retrieval member 108 is displaced proximally and/or pulled toward the cervix.

In embodiments retrieval member 108 may be provided from optional material for example including but not limited to at least one or more of flexible polymeric material, Polypropylene, High-Density Polyethylene (HDPE), Nylon and the like.

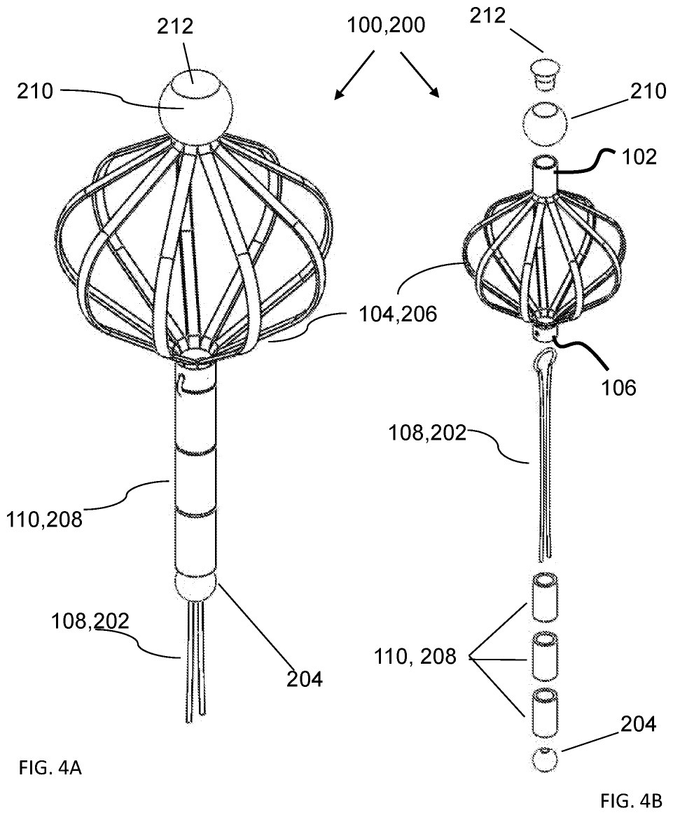

FIG. 4A illustrates a non-limiting embodiment of IUD device 100 in the form of an intrauterine 3-dimensional device 200 that is intended to be used as a long-acting reversible contraceptive (LARC) in accordance with some embodiments of the present invention.

The intrauterine 3-dimensional device 200 comprises a retrieval member in the form of strings 202, self-expandable anchor 206, an active agent module 110 in the form of copper such as at least one copper sleeve 208 and distal copper bead 210.

The intrauterine 3-dimensional device 200 further comprises proximal body portion 106 that is associated with a proximal stopper 204, and a distal body portion 102 associated with a distal stopper 212.

In embodiments a vaginal speculum and medical imagery devices, for example including but not limited to ultrasound, as are known in the art, may be utilized to facilitate delivery and/or deployment of the device 200 into the uterus and the uterine cavity.

FIG. 4B shows an exploded view of FIG. 4A showing the intrauterine 3-dimensional device 200 in accordance with some embodiments of the present invention.

As shown proximal stopper 204, the copper sleeves 208, the strings 202, the self-expandable anchor 206, the distal copper bead 210, and the distal stopper 212. In accordance with some embodiments of the present invention, the distal stopper 212 may be made of various materials such as metal, alloy, and polymer(s), for instance, Polyethylene.

In accordance with some embodiments of the present invention, treatment module 110 provided in the form of copper bead 210 and/or the copper sleeves 208 may be made of copper having, for instance, a purity of at least 99.99% with a total surface area of 150 to 380 mm2 (millimeter squared) and preferably 200 to 380 mm2 (millimeter squared).

In accordance with some embodiments of the present invention, the proximal stopper 204 threaded and secured to the strings 202 may be design as a bead or sleeve made of metal, Silicone, or polymeric material such as Polyurethane.

In accordance with some embodiments of the present invention, the strings may be made of flexible polymeric material such as Polypropylene, High-Density Polyethylene (HDPE), Nylon and the like.

In some embodiments as previously described treatment module 110 may be provided in the form of a hormone reservoir instead of copper sleeves 208 and/or copper bead 210 associated with of the intrauterine 3-dimensional device 200 shown in FIG. 4A-B.

In embodiments, the device according to the present invention, as depicted and described in FIG. 4A-B may be assembled prior to delivery in the following manner, with reference to FIG. 4B showing an exploded view of the device 200. Accordingly, a functional treatment bead 210 may be disposed and/or functionally associated about the distal end 102 of device 200. In embodiments treatment bead 208, 210 is configured to comprise at least one or more of active agents and/or medicament for example including but not limited to copper and/or a hormone, chemical agent, drug medicament, any combination thereof or the like.

Next the distal stopper 212 may be utilized to affixed onto the functional treatment bead 210. In embodiments the distal stopper may be affixed via press fit, mechanically, male female connectors, gluing, chemical interaction, welding, the like or any combination thereof.

In embodiments, strings 202 is threaded through the proximal opening of the self-expandable anchor 206. Followed by treatment sleeve treatment member 110 that is associated proximally to the anchor 206 over the string 202. In embodiments sleeve treatment member may for example comprise a copper sleeve 208, hormones reservoir or the like treatment vector and/or medicament. Next proximal stopper 204 is disposed proximally along string 202 proximal to treatment sleeve member 208,110 and is secured thereto by optional means for example including but not limited to male/female coupling, friction fit, welding, knot or the like.

FIG. 5A-B illustrate a non-limiting embodiment of IUD device 100 featuring an agent delivery channel assembly 130, therein depicted in the form of an intrauterine 3-dimensional device 800 that is used for short-term fluids delivery into the uterus in accordance with some embodiments of the present invention.

FIG. 5B is an exploded view and FIG. 5A is a cross-sectional view of an intrauterine 3-dimensional device 800 used for short-term fluids delivery into the uterus in accordance with some embodiments of the present invention.

The intrauterine 3-dimensional device 800 comprises a balloon catheter 804, balloon 806 (deflated in FIG. 5B and inflated in FIG. 5A), an eyelet 808, self-expandable anchor 810, a thin layer seal 812, a proximal stopper 814, a self-sealing septum 822 and a flexible dripping cannula 802.

In accordance with some embodiments of the present invention, the balloon catheter 804 may have a preferred catheter size of 4 Fr (1.35 mm) to 7 Fr (2.30 mm). The balloon 806 is adhered to the catheter 804 and covers an eyelet 808 trimmed at the distal end of the catheter 804. The balloon 806, is disposed along an internal volume of the support structure 810 may be inflated by a sterile fluid through the proximal end of the catheter 804 and eyelet 808. In this configuration the inflated balloon 806 is pressurized (low pressure), so the internal pressure is higher than external atmospheric pressure (FIG. 5A).

In accordance with some embodiments of the present invention, the flexible shaft of the catheter 804 may be made of flexible polymeric material such as Polyurethane or Silicone and the thin wall balloon 806 may be made of flexible polymeric material(s) such as Polyurethane, Nylon, PET, EVA membrane or elastomeric material such as Silicone, Silicone membrane PDMS (polydimethylsiloxane), Polyurethane and the like.

In accordance with some embodiments of the present invention, the flexible cannula 802 may be connected to the balloon catheter's distal outlet 818, and the balloon's wall may be drilled for some micro-holes by laser or similar means, so when the elastic balloon 806 is inflated with fluid, for example including but not limited to washed sperm, sperm, drug, active agent, biologics, any combination thereof or the like, it functions as a small volume elastomeric pump to drip liquid droplets locally inside the uterus, for instance, in a slow-release manner, for example as shown in FIG. 5A.

In accordance with some embodiments of the present invention, the flexible dripping cannula 802 may be an extruded flexible tube made of thermoplastic elastomer (TPE) such as Pebax, Polyurethane or Polyethylene.

In accordance with some embodiments of the present invention, the flexible dripping cannula 802 pre-defines the flow rate controlled by parameters such as inner diameter, length, and friction.

In accordance with some embodiments of the present invention, the flexible dripping cannula 802 is in communication with the balloon's pressurized volume and functions as a flow restrictor in elastomeric pump, FIG. 5A.

In accordance with some embodiments of the present invention, the proximal stopper 814 may be a protrusion made of Silicone, Polyurethane and the like and adhered to catheter shaft of the catheter 804.

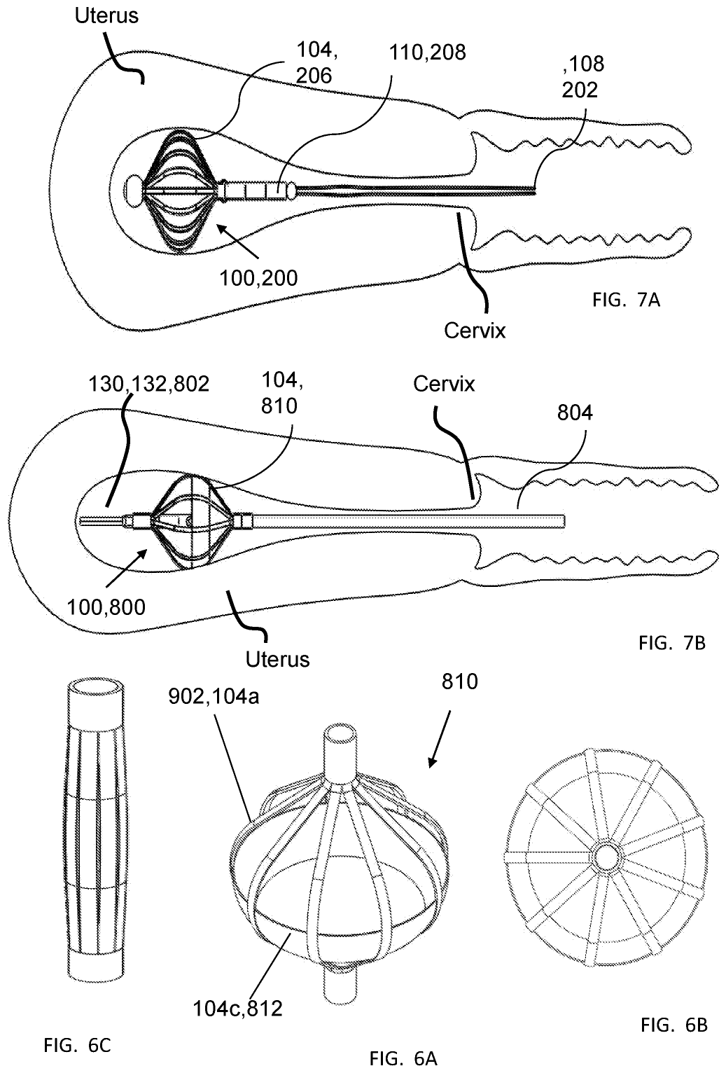

FIGS. 6A-C illustrate a close-up view of the self-expandable anchor 810 of the intrauterine 3-dimensional device 800 in accordance with some embodiments of the present invention.

FIG. 6A is a perspective view of the self-expandable anchor 810.

FIG. 6B is top view of the self-expandable anchor 810 in an expanded configuration.

FIG. 6C illustrates the self-expandable anchor 810 in a collapsed configuration.

In accordance with some embodiments of the present invention, the self-expandable anchor 810 may be a slotted-tube made of super-elastic and/or shape-memory materials for example including but not limited to Nitinol (NiTi; a nickel-titanium alloy). The slotted-tube may undergo heat treatment to form the final shape of the self-expandable anchor 810, e.g., 3-dimensional multiple ribs 902,104a sphere as seen in FIGS. 6A-C.

In accordance with some embodiments of the present invention, at least a portion of anchor 810 for example the lower half of the self-expandable anchor 810, as shown in FIG. 6A-B may be coated by a thin layer seal 812 made of elastomeric material such as Silicone or polymeric material(s) to have a design like inverted umbrella, for example as shown in FIG. 6A.

In embodiments, seal 812 or the like coating 104c of a portion of anchor 810,104 may be utilized to ensure the directionality of fluid flow. For example, as described in FIG. 5A-B, when fluid is delivered via a distal catheter 132,802, seal 812 or the like coating 104c may be utilized to ensure that fluid flow is not retrograde and/or proximal so as to minimize and/or prevent loss of flowing fluid delivered via distal catheter 802,132.

In accordance with some embodiments of the present invention, the balloon 806, is preferably disposed along the internal volume of anchor 810 and may be inflated by a flowing fluid for example including but not limited to washed sperm, sperm, biologics, active agent, hormone, drug, or medicine, the like or any combination thereof. The flexible dripping cannula 802 is preferably configured to be in fluid communication with balloon's pressurized volume. In such configuration the inflated balloon 806 is pressurized (low pressure), so internal pressure is higher than atmospheric pressure and fluid is dripping inside the uterine cavity in a slow-release manner due to differential pressure.

In embodiments, as shown in FIG. 5A-B is arranged in distal to proximal arrangement as is shown in the exploded view of FIG. 5B wherein care is taken to ensure that a flow path between balloon 806 and cannula 802 is provided in a unidirectional manner, so that fluids do not leak proximally, from the proximal end of catheter 804.

In embodiments, balloon 806 may be introduced into the internal volume of anchor 810 via proximal body member 816

FIG. 7A-B show a sectional perspective view of a uterus that is fit with optional configuration of IUD device 100 according to embodiments of the present invention.

FIG. 7A shows IUD device 100, 200 in its deployed, in use, configuration within the uterine anatomy. A shown, anchor portion 206, 104 abuts the uterine tissue, while retrieval member 108,202 is disposed across the length of the cervix, extending external to the uterus to facilitate removal. In embodiments, device 100,200 features a treatment module 110,208 that may be configured for long term use for example for reversible contraceptive use.

FIG. 7B shows IUD device 100, 800 featuring an agent delivery assembly 130, in its deployed, in use, configuration within the uterine anatomy. A shown, anchor portion 810, 104 abuts the uterine tissue while fluid delivery catheter 802,132 are disposed distal to anchor portion 810 so as to facilitate local delivery of an optional fluid, as previously described. In embodiments catheter 804 may be utilized to introduce the treatment flowing fluid and to inflate a balloon 806 (not shown here). In embodiments, device 100 featuring agent delivery assembly 130 may be configured for time-limited use for the purpose of delivery the treatment and/or fluid through the distal catheter 802, 132, and therefore configured for short term use based on the duration of the treatment.

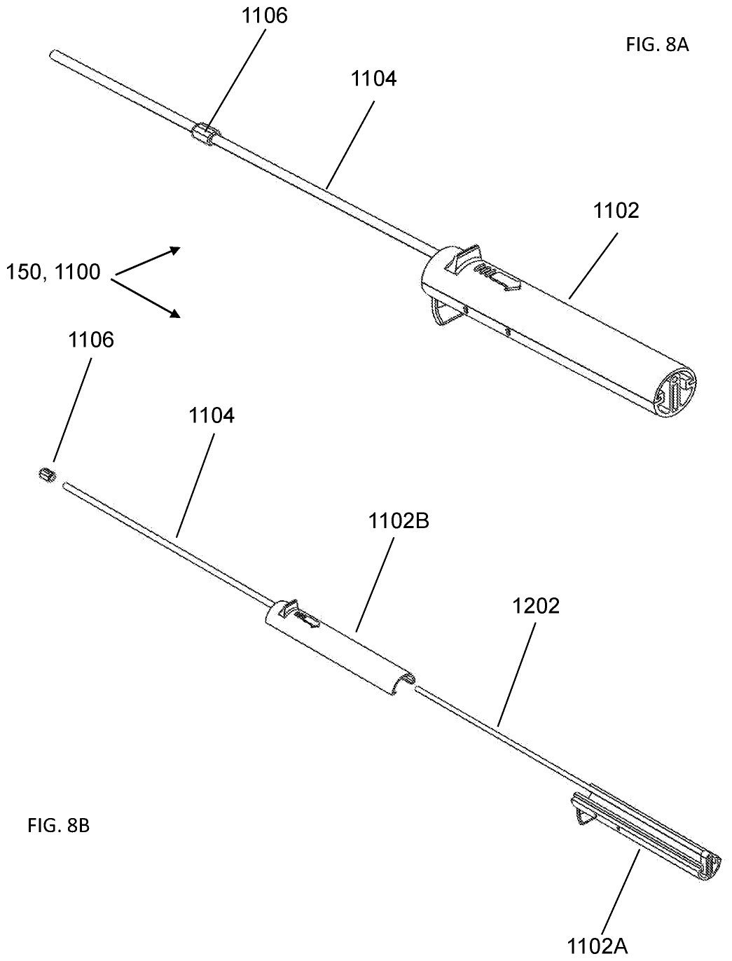

Now referring to FIG. 8A-B showing a dedicated tool 150,1100 configured to introduce IUD device 100 within the uterine anatomy, as shown in FIG. 7A-B. In embodiments introducing tool 150,1100 provides for deploying and/or inserting device 100 through the cervix in small profile configuration 100a, and further allowing device 100 to assume its expanded configuration 100b, after delivery within the uterus.

In embodiments, inserter 1100 is configured for inserting the intrauterine 3-dimensional device 200, 800 into the uterus in accordance with some embodiments of the present invention.

As shown, the inserter 1100 comprises a handle 1102, an insertion tube 1104 and a flange 1106. Preferably, handle 1102 comprises two halves, upper part 1102B and lower part 1102A, when the upper part 1102B is preferably configured to slide over the lower part 1102A. The insertion tube 1104 is functionally associated with to the upper part 1102B and the plunger 1202, shown in FIG. 8B, is functionally associated with the lower part 1102A. The flange 1106 is mounted over the insertion tube 1104 and can be adjusted along it as required. Preferably flange 1106 is configured to provide a depth measuring gage.

FIG. 8B shows an exploded view of the inserter 1100 depicted in FIG. 8A, revealing lower slider 1102A and the upper slider 1102B of handle 1102, a plunger 1202, the insertion tube 1104 and the flange 1106.

In accordance with some embodiments of the present invention, the handle 1102 may be made of metal, alloy, or a polymeric material such as, for instance, ABS, Polycarbonate or Polypropylene.

The plunger 1202 may be an extruded flexible tube made of metal, alloy, or a polymeric material such as, for instance, ABS or stainless steel.

The flange 1106 may be made of metal, alloy, or a polymeric material such as, for instance, Polyethylene or Polypropylene.

The insertion tube 1104 may be an extruded flexible tube made of materials such as metal, alloy, or a polymeric material such as, for instance, Polyethylene or Polypropylene. The distal part of the insertion tube 1104 may be marked on its external surface with ruler scale to indicate the insertion depth into the uterus.

FIG. 8B showing an exploded view of inserter 1100 provides a distal to proximal arrangement of the inserter 1100,150.

In embodiments at least a distal portion of plunger 1202 is configured to be aligned with the insertion tube 1104. Most preferably, insertion tube 1104 is configured to receive and house IUD device 100, 200 800 prior to delivery within the uterus. Preferably insertion tube 1104 is configured to retain device 100 in its low-profile configuration 100a prior to and during delivery.

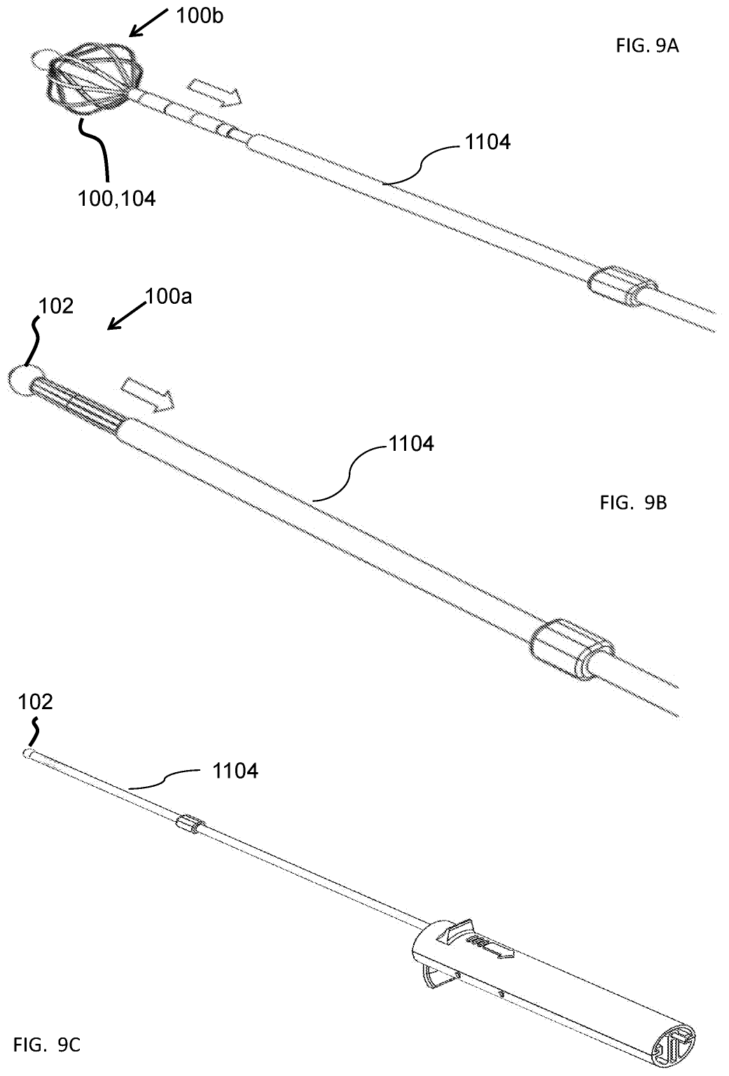

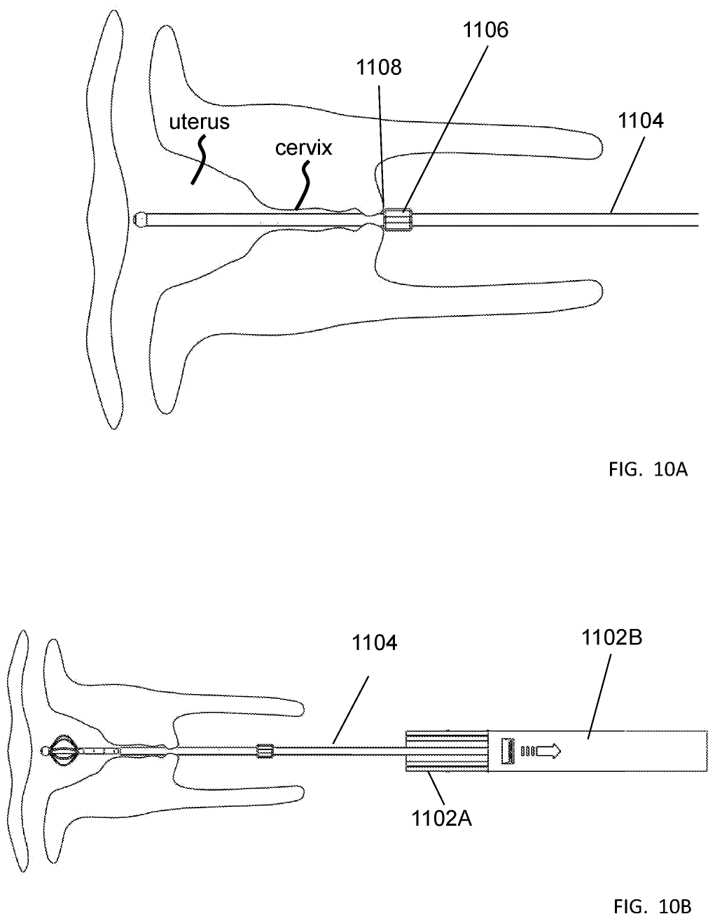

FIG. 9A-C show the process of associating an IUD device 100 according to embodiments of the present invention with introducing device 150,1100. As shown in FIG. 9A an optional IUD device 100 is introduced into the lumen of insertion tube 1104. FIG. 9B shows IUD device 100 that is partially associated within the internal lumen of insertion tube 1104 wherein self-expanding body portion 104 is partially collapsed as it moves proximally into insertion tube 1104, for example a shown with the directional arrow. FIG. 9C shows IUD device 100 that is wholly associated within the lumen of insertion tube 1104, wherein in preparation for delivery through the cervix only the distal end portion 102 of device 100 extends from tube 1104.

FIG. 10A-B provides an illustrative depiction of the delivery process of device 100 with dedicated device 150, 1100 where IUD device 100 is deployed and/or inserted through the cervix and into the uterus where it assumes its expanded configuration 110b.

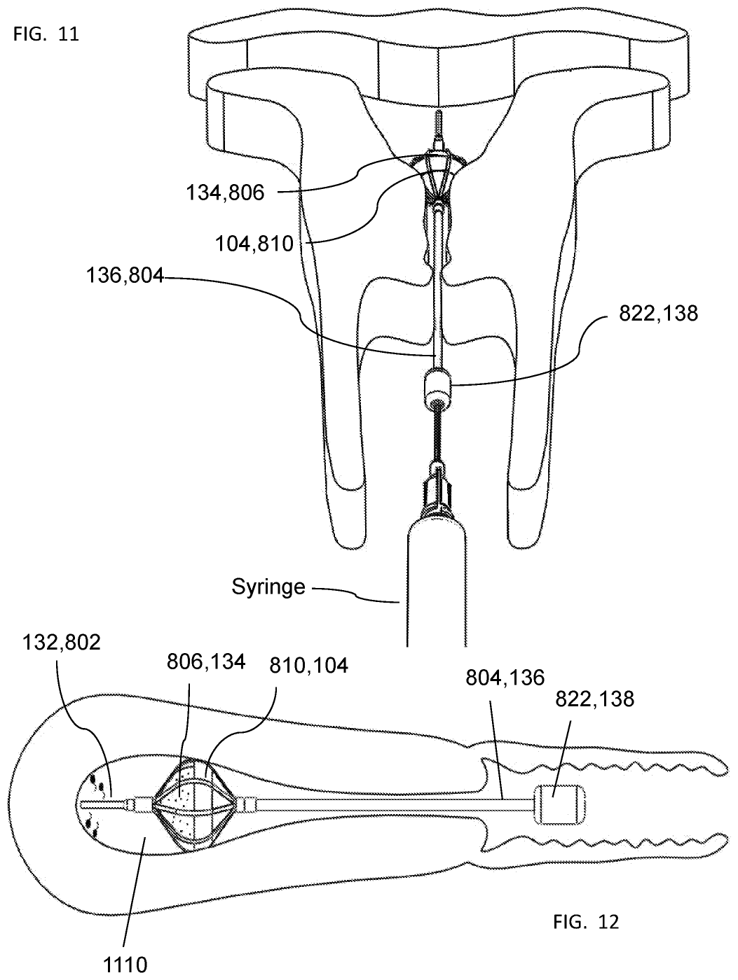

Now referring to FIG. 11-14, showing embodiments relating to IUD device 100 featuring a fluid agent delivery assembly 130, as previously described and shown at least with respect to FIG. 5A-B with respect to IUD device 800.

FIG. 11-12 show IUD device 100,800 in use within the uterine anatomy. FIG. 11 shows device 100,800 placed within the uterus where balloon 134,806 is inflated by introducing a fluid to catheter 136,804 via a proximal end of sealing member 138,822 therein a fluid external to the uterine anatomy is introduced and delivered at a distal catheter 132,802.

As shown in FIG. 11 balloon 134,806 is inflated with a fluid introduced with a syringe. As shown in FIG. 12, fluid delivery, for example shown in the form of fertilization fluid and/or sperm, is delivered locally within the uterus at the distal cannula and/or channel 132,802. As shown, a proximal end catheter 804 is fit with a sealing member 138,822 that is utilized to ensure that the treatment fluid is delivered distally and is not lost proximally, as sealing member 138,822 seals catheter 804 to ensure that the proximal end of catheter 804 is sealed and there is no retrograde fluid flow.

In embodiments sealing member 138,822 is introduced onto proximal end catheter 136,804 utilizing a dedicated sealing member introducing tool 1600,140, shown in greater detail in FIG. 13-14.

FIG. 13-14 show a non-limiting example of sealing member introducing tool 1600,140 according to embodiments of the present invention.

FIG. 13A-C show various views of introducing tool 1600,140 in use while utilized for introducing self-sealing septum 822. FIG. 13A shows a perspective view,

FIG. 13B shows an end view of the distal end. And FIG. 13C shows a partial exploded view of a dedicated sealing member introducing device 1600,140, also interchangeably referred to as septum applicator.

As best seen in FIG. 13C, sealing member 822 is associated with the septum applicator tool 1600 that comprises a handle 1602, tracks featuring female tracks 1604A and a corresponding male tracks 1604B, flexible arms 1606, cavity and/or recess 1616 for receiving and/or holding septum sealing member 822, an integral hinge 1610, a funnel shaped cannula 1612, and slider housing member 1614. In embodiments, handle 1602 features a receiving recess and/or cavity 1616 at the distal end of handle 1602 that is configured to retain the self-sealing septum 822 in place. Preferably the two flexible arms 1606 are configured to retain and/or hold sealing member 822 within recess 1616 by urging and/or pressing member 822 inwardly and/or proximally internal to recess 1616. In embodiments, slider housing member 1614 is preferably functionally associated with and/or attached to a funnel shaped cannula 1612, that provides for facilitating association of sealing member 822 with the distal end of catheter 804, as will be shown in greater detail in FIG. 14A-E. In embodiments, funnel shaped cannula 1612 comprises two longitudinal portion and/or halves 1612A, 1612B that are integrally attached to one another with an integrated hinge 1610, as best seen in FIG. 14B-14C. Integrated hinge member 1610 provides for closing funnel shaped cannula 1612 or for opening cannula 1612 about hinge 1610 therein distancing and/or separating its two integrated halves 1612A, 1612B respectively along one edge opposite hinge 1610, along the long axis of funnel shaped cannula 1612.

In embodiments, funnel shaped cannula 1612 in its closed configuration, FIG. 13A, occurring when its two halves 1612A, 1612B, are folded about hinge 1610 and coupled an/or associated with one another, along the long axis. Accordingly, in its folded and/or closed configuration cannula 1612 comprises a distal funnel shaped portion 1612D and a proximal channel/tubular portion 1612C, FIG. 13B. In embodiments funnel shaped portion 1612D provides for introducing catheter 804 into the internal volume and/or lumen of funnel shaped cannula 1612, as best seen in FIG. 14A-14B. In embodiments, proximal tubular shaped portion 1612C, provides for functionally coupling funnel shaped cannula 1612 with sealing member 822 along an internal lumen 824, FIG. 13C and FIG. 14A, wherein proximal portion 1612C is disposed internal to the lumen 824 of sealing member 822. In embodiments, funnel shaped cannula 1612 is preferably pushed proximally into the lumen 824 of self-sealing septum 822, to optionally and preferably expand its internal bore/lumen 824, so as to prepare and/or ready it for introducing and/or receiving at least a portion of a flexible catheter, for example catheter 804, into the lumen 824 of septum 822.

In accordance with some embodiments, the handle 1602 may be made of optional materials for example including but not limited to metal, alloy, or polymeric material(s) such as, for instance, ABS, Polycarbonate or Polypropylene, the like or any combination thereof.

In embodiments, self-sealing septum 822,138 may be an elastomeric component pre-slit made of an elastomeric material for example including but not limited to Silicone, polymeric material(s) such as Polyurethane, the like or any combination thereof.

In embodiments, self-sealing septum 822,138 may comprise at least one or more dedicated portion, for example port 826, that is provided from elastomeric component forming a pre-slit injection site made of an elastomeric material for example including but not limited to Silicone, polymeric material(s) such as Polyurethane, the like or any combination thereof. In embodiments such pre-slit injection site provides a multi-use injection site which will reliably reseal even after many insertions with a blunt cannula, syringe or the like.

In embodiments, slider 1614 may be made of optional materials for example including but not limited to metal, alloy, or a polymeric material such as, for instance, Polyethylene, Nylon, Polypropylene and the like.

The funnel shape cannula 1612 may be functionally coupled to and/or connected to the slider 1614 by optional means for example including but not limited to welding, gluing, mechanical snap-fit or any other way mechanical coupling means.

FIG. 14A-E depict a method for loading a self-sealing septum 822 onto a corresponding septum applicator tool 1600 in preparation for use. Preferably septum 822 is disposed internal to applicator 1600 particularly inside receiving recess and/or cavity 1616 at the distal end of the handle 1602, for example as shown in FIG. 13B-13C. Preferably, slider 1614 is associated over handle 1602 and directing it into the appropriate tracks, for example including female tracks 1604A and male tracks 1604B on either side of the handle 1602. Preferably two male tracks 1604B of the slider 1614 are directed into the appropriate female tracks 1604A of the handle 1602 to obtain an assembled applicator. Then, to complete the sliding movement, pressing the two flexible arms 1606 inwards and locking the septum 822 in place by their two protruded teeth 1606C, for example as shown in FIG. 13A,14A. Preferably, due to its elasticity, septum 822 can be stretched radially outwards so as to accommodate the funnel shape cannula 1612, at least about proximal portion 1612C within lumen 824. Once funnel cannula 1612 is disposed within the lumen 824 of septum 822, as shown in FIG. 13B,14A, cannula 1612 is configured to facilitate association with catheter 804 of intrauterine 3-dimensional device 800, wherein catheter end 804 is disposed internal to septum canal 824 of septum 822 therein utilized to block the distal end of catheter 804, as previously described and shown in FIG. 5A-B,

FIG. 14A shows a sectional view of applicator 1600 that has been associated with the distal end of catheter 804 wherein the distal end has penetrated the lumen of funnel shaped cannula 1612 along both the proximal portion 1612C and distal portion 1612D that reside internal to lumen 824 of sealing member 822.

FIG. 14B shows a cross sectional view showing the initiation of applicator tool 1600 from sealing member 822 and catheter 804 so as to allow sealing member 822 to couple with the distal end of 804 therein to seal catheter 804. Accordingly, as shown in FIG. 14B, in order to remove and/or disassociate applicator 1600 first slider 1614 is displaced distally (forward) as shown by the directional arrow. This forward movement allows funnel shaped cannula 1612 to disassociate from catheter 804 while allowing sealing member 822 to be released onto catheter. Preferably, the forward motion is provided while gripping catheter 804 in place, until funnel shaped cannula 1612 is fully released out of the lumen 824 of sealing member 822. Once funnel shaped cannula 1612 is released from sealing member 822, cannula 1612 is opened about its integrated hinge 1610, so that it assumes its open configuration wherein first portion 1612A and second portion 1612B are disassociated about hinge 1610, as shown with the curved directional arrow in FIG. 14B.

Next, once funnel shaped cannula 1612 is in the open configuration, the applicator's slider housing member 1614 may be dissociated from applicator 1600, for example a shown in FIG. 14C.

Next, as shown in FIG. 14D, applicator handle 1602 is displaced proximally, as shown with the directional arrow, to release sealing member 822 from applicator tool 1600. As handle 1602 is displaced proximally flexible arms 1606, first flexible arm 1606A and second flexible arm 1606B release their hold of sealing member 822 as the separate and become wider outwardly, as shown in FIG. 14D. In embodiments, as arms 1606A,B become wider outwardly, the user can retract the handle 1602, leaving the septum 822 mounted tightly onto the catheter 804 to obtain sealed connection therebetween, as is shown in FIG. 14E.

In some embodiments, the movement of both slider 1614 forward distally is performed simultaneously with the proximal displacement of handle 1602 such that the release of seal member 822 occurs in substantially a single motion.

FIG. 14E shows a sectional view of associated sealing member 822 with distal end of catheter 804 as previously described in at least FIG. 5A-B. As shown here sealing member 822 features an internal lumen 824 that is configured to secure over a distal end of catheter 804 to seal its. Sealing member 822 further features a proximal end 826 provides a port allowing for fluid delivery therethrough onto catheter 804 as previously described, for example with a syringe. Optionally and preferably proximal end port 826 is a self-sealing port configured for allowing unidirectional flow therethrough wherein a fluid may be introduced via port 826, for example with a syringe of the like, but does not allow fluids to flow in the opposite direction therein ensuring that the distal end of catheter 804 remains sealed fluid delivery is established. Optionally, balloon 806,134, as previously described and shown, may be inflated and/or deflated with a flowing fluid via sealing member proximal end port 826. In embodiments, port 826 may serve to introduce any flowing fluid into device 100,800, as previously described, for example to a distal end catheter 802,132.

As used herein the term “about” refers to +/−10%.

The terms “comprises”, “comprising”, “includes”, “including”, “having” and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

There are many inventions described and illustrated herein. The present inventions are neither limited to any single aspect nor embodiment thereof, nor to any combinations and/or permutations of such aspects and/or embodiments. Moreover, each of the aspects of the present inventions, and/or embodiments thereof, may be employed alone or in combination with one or more of the other aspects of the present inventions and/or embodiments thereof. For the sake of brevity, many of those permutations and combinations will not be discussed separately herein.

While the invention has been described with respect to a limited number of embodiment, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not described to limit the invention to the exact construction and operation shown and described and accordingly, all suitable modifications and equivalents may be resorted to, falling within the scope of the invention.

It should be noted that where reference numerals appear in the claims, such numerals are included solely or the purpose of improving the intelligibility of the claims and are no way limiting on the scope of the claims.

Having described a specific preferred embodiment of the invention with reference to the accompanying drawings, it will be appreciated that the present invention is not limited to that precise embodiment and that various changes and modifications can be effected therein by one of ordinary skill in the art without departing from the scope or spirit of the invention defined by the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the invention.

Section headings are used herein to ease understanding of the specification and should not be construed as necessarily limiting.

While the invention has been described with respect to a limited number of embodiments, it will be appreciated that many variations, modifications and other applications of the invention may be made.

1. An intrauterine 3-dimensional device (100,200,800) comprising:
a. a self-expandable anchor (104) portion provided from a slotted-tube made of super-elastic or shape-memory material;
the self-expandable anchor having an elastic three-dimensional spherical structure formed from a plurality of ribs (104a), wherein when the self-expandable 3-dimensions shape anchor is deployed inside the uterus and radially expanded, said 3-dimensions shape anchor has peripheral retention against the uterus wall to prevent displacement and/or migration and/or expulsion of the intrauterine 3-dimensional device out of the uterus;
b. a pulling member (108) functionally coupled to a proximal end (106) of the self-expandable anchor (104) to enable pulling and removal of the intrauterine 3-dimensional device out of the uterus;
wherein the anchor (104) is axially expanded to assume a wider structure (100b) to resist expulsion from the uterus when being pushed by the uterus forces; and
wherein the anchor is radially collapsible to assume a narrow configuration (100a) to allow removal from the uterus when being pulled out with said pulling member (108).

2. (canceled)

3. The device of claim 1, wherein a rounded distal tip (210) is connected to a distal end (102) of the self-expandable anchor.

4. The device (100,800) of claim 1 configured for delivery of a flowing fluid into the uterus, the device further comprising a fluid flow delivery assembly (130) that features:
a. a proximal catheter (804,136);
b. a compliant balloon (806,134) enclosed within an internal volume (104v) of said self-expandable anchor (810,104); wherein said balloon is connected to a distal end (818) of said catheter (804,186);
c. a sealing septum (822,138) connected to a proximal end of said catheter (136,804), said sealing septum (822) is penetrable for bidirectional flowing flow between said catheter (804) and said balloon (806);
d. a fluid flow delivery cannula (802,132) in fluid communication with said distal end (818), wherein said fluid flow delivery cannula and said balloon have a single, common fluid flow path, wherein when the balloon is inflated with a fluid, the balloon is configured to delivery fluid droplets via said fluid flow delivery cannula (822,132).

5. The device of claim 4 wherein said fluid flow delivery cannula (802) is configured in order to control the fluid flow rate therethrough according to at least one cannula parameter selected from inner diameter, length, friction, friction coefficient, number of micro-holes, number of micro-pores, size of micro-holes, size of micro-pores, pore diameter, pore shape, or any combination thereof.

6. (canceled)

7. The device of claim 4, wherein said sealing septum (822) comprises a proximal end filling port (826) configured to provide an access point configured to provide bidirectional fluid flow communication between at said catheter (804) and said balloon (806).

8. The device of any one of claim 4, wherein the sealing septum (822) features a self-sealing pre-slit inlet.

9. The device of claim 4, wherein said catheter (804) is integrated with said balloon (806) and features an eyelet (808) trimmed at the distal end of the catheter, said eyelet (808) configured of controlling bidirectional fluid flow through said balloon (806).

10. (canceled)

11. (canceled)

12. The device of any one of claims 1 further comprising a treatment module (110) associated with at least a portion of said device; wherein said treatment module comprises an active agent selected from copper, hormone reservoir, chemical substance, biological substance, drug, medicament, a copper sleeve, a copper containing solid, a copper containing alloy, and/or copper bead or any combination thereof.

13. (canceled)

14. (canceled)

15. The device of claim 12, wherein the selected active agent is copper having a surface area of 150 mm^2 up to 380 mm^2 (squared millimeters).

16. The device of any one of claim 1 further comprising an elastomeric sealant (104c,812) configured to cover at least a portion of said anchor (810,104).

17. (canceled)

18. (canceled)

19. (canceled)

20. The device of claim 4, wherein said flowing fluid is selected from the group comprising injectables, sperm, chemical agent, biological agent, hormone, drug, and medicine, or any combination thereof.

21. The device of claim 4, wherein the cannula is an extruded flexible tube flow restrictor configured to control the fluid delivery rate.

22. (canceled)

23. (currently amendment) The device of any one of claims 1, further comprising a sensor or biosensor.

24. (canceled)

25. (currently amendment) The device of any one of claims 23, wherein said sensor or biosensor is selected from the group consisting of at least one of pH sensor, temperature sensor, pressure sensor, electrolytes sensor, oxygen concentration sensor, or any combination thereof.

26. A kit for reversible contraception and/or for active agent delivery and/or for insemination of a human or animal female, the kit comprising:
a. an IUD device according to claim 1;
b. a dedicated IUD insertion tool (1100,150) configured for inserting and positioning said IUD device within the uterine cavity;
c. a sealing member introducing tool (1600,140) featuring a funnel shape cannula (1612) having two halves (1612A,1612B) that are coupled with an integrated hinged (1610); wherein said sealing member introducing tool (1600) is configured for mounting a self-sealing septum (822) on a proximal end of said catheter (804).

27. (canceled)

28. The kit of claim 26, wherein the septum applicator (1600) further comprises a handle (1602), tracks (1604, 1604A, 1604B), flexible arms (1606), a sealing member receiving recess (1616), and a slider (1614).

29. (canceled)

30. The kit of claim 26 further comprising a sealing septum (822).

31. (canceled)

32. The kit of claim 26, wherein the dedicated IUD insertion tool (1100) comprises a handle (1102), an insertion tube (1104), a plunger (1202), and a flange (1106).

33. The kit of claim 32, wherein the distal part of said insertion tube further comprises marking along its external surface providing ruler scale indicative of an insertion depth into the uterus.

34. The device of claim 1, wherein said super-elastic or shape-memory material is selected from a nickel-titanium alloy or a shape-memory polymers or any combination thereof.

35. (canceled)
